(19) United States
(12) Patent Application Publication
(10) Pub. No.: US 2022/0154132 A1
(43) Pub. Date: May 19, 2022

(54) METHOD OF REDUCING CONCENTRATIONS OF ONE OR MORE OF N2O and NO IN MEDIUM

(71) Applicant: Samsung Electronics Co., Ltd., Suwon-si (KR)

(72) Inventors: Seung Hoon Song, Suwon-si (KR); Woo Yong Shim, Suwon-si (KR); Jae-Young Kim, Suwon-si (KR); Yu Kyung Jung, Hwaseong-si (KR)

(21) Appl. No.: 17/223,340

(22) Filed: Apr. 6, 2021

(30) Foreign Application Priority Data
Nov. 17, 2020 (KR) 10-2020-0154090

(51) Int. Cl. C12N 1/20

(52) U.S. Cl. CPC C12N 1/20

(57) ABSTRACT

A method of reducing a concentration of N₂O, NO, or a combination thereof in a medium, the method comprising: culturing a microorganism of the genus Paracoccus, a microorganism of the genus Pseudomonas, or a combination thereof in a liquid medium comprising Mg²⁺ ions and Fe(II)(L)-NO, N₂O, or a combination thereof, wherein L is a chelating agent; and reducing NO to N₂O or N₂, or reducing N₂O to N₂.

### CROSS-REFERENCE TO RELATED APPLICATION

This application claims priority to and the benefit of Korean Patent Application No. 10-2020-0154090, filed on Nov. 17, 2020, in the Korean Intellectual Property Office, and all the benefits accruing therefrom under 35 U.S.C. § 119, the content of which in its entirety is incorporated herein by reference.

### BACKGROUND

### 1. Field

The present disclosure relates to a method of reducing a concentration of N₂O or NO in a medium.

### 2. Description of the Related Art

The release of a nitrogen oxide gas such as N₂O and NO into the atmosphere is a significant environmental problem. Nitrogen oxides are generally referred to as NOx. Ozone depletion, climate warming, and acidification of soil and water systems are each attributed to the emission of nitrogen oxide gas.

There remains a need for an alternative method, which is capable of efficiently removing a nitrogen oxide, such as N₂O and NO.

### SUMMARY

An aspect provides a method of reducing a concentration of one or more of N₂O and NO in a medium, the method including reducing NO to N₂O or N₂ or reducing N₂O to N₂ by culturing a microorganism (bacterium) of the genus Paracoccus or a microorganism of the genus Pseudomonas in a liquid medium containing Mg²⁺ ions and Fe(II)(L)-NO or N₂O, wherein L is a chelating agent.

Additional aspects will be set forth in part in the description which follows and, in part, will be apparent from the description, or may be learned by practice of the presented embodiments of the disclosure.

An aspect provides a method of reducing a concentration of N₂O, NO, or a combination thereof in a medium, the method including: culturing a microorganism of the genus Paracoccus, a microorganism of the genus Pseudomonas, or a combination thereof in a liquid medium including Mg²⁺ ions and Fe(II)(L)-NO, N₂O, or a combination thereof, wherein L is a chelating agent; and reducing NO to N₂O or N₂, or reducing N₂O to N₂.

In the method, the microorganism of the genus Paracoccus may include Paracoccus versutus, Paracoccus denitrificans, Paracoccus pantothrophas, Paracoccus ferrooxidans, and Paracoccus denitrificans. The microorganism of the genus Pseudomonas may be selected from the group consisting of Pseudomonas stutzeri, Pseudomonas putida, Pseudomonas cepacia, Pseudomonas fluorescens, Pseudomonas mendocina, or a combination thereof.

In the method, the liquid medium may include about 0.1 millimolar (mM) to about 7.5 mM, about 0.5 mM to about 7.5 mM, about 0.5 mM to about 5.0 mM, about 0.5 mM to about 2.5 mM, about 0.5 mM to about 1.5 mM, or about 1.0 mM to about 2.5 mM of Mg²⁺ ions.

In the method, Fe(II)(L)-NO represents a complex formed by chelating the chelating agent L with Fe²⁺ and NO. The L may be, for example, ethylenediamine, diethylenetriamine, triethylenetetraamine, hexamethylenetetraamine, N-(2-hydroxyethyl)ethylenediamine-triacetic acid (HEDTA), ethylenediamine-tetraacetic acid (EDTA), iminodiacetic acid, nitrilotriacetic acid (NTA), or diethylenetriaminepentaacetic acid (DTPA).

The liquid medium may be a medium that enables the growth of the microorganisms. The culturing may be performed under anaerobic conditions. The culturing may be performed at a temperature of about 25° C. to about 40° C., or about 25° C. to about 35° C.

The culturing may include culturing of the microorganism of the genus Paracoccus, culturing of the microorganism of the genus Pseudomonas alone, or culturing a mixture of the microorganism of the genus Paracoccus and the microorganism of the genus Pseudomonas. The culturing may be performed under conditions that allow the microorganisms to proliferate (grow) or to maintain viability. The culturing may be performed in a medium and at temperature conditions that facilitate proliferation of the microorganism. The culturing may be performed with stirring.

The method may not include an additional denitrification process other than the culturing. The method converts N₂O or NO into N₂ by the culturing, and may not include an additional biological denitrification process.

In the method, the method further includes forming of Fe(II)EDTA-NO by contacting a nitrogen oxide, e.g., NO₂ or NO, with a liquid medium containing Fe(II)EDTA. The culturing may be performed at the same time as the forming of Fe(II)EDTA-NO or after the forming of Fe(II)EDTA-NO in the medium. The concentration of Fe(II)(L)-NO in the medium may be about 1 mM to about 200 mM, about 25 mM to about 150 mM, or about 0.1 mM to 20 about mM.

In addition, the liquid medium may further include an electron donor. The electron donor may be an organic carbon compound. The electron donor may be methanol, ethanol, acetate, lactate, citrate, glucose, sucrose, or a combination thereof.

The liquid medium may be a growth medium or a buffer. The liquid medium may be a chemically defined medium. As used herein, the term “chemically defined medium” refers to a medium in which the chemical components and their corresponding concentrations are known. The chemically defined medium may be a medium that does not include a complex component such as, for example, serum or a hydrolysate. The liquid medium may include an Luria Delbruck (LB) medium, an M9 medium, a phosphate buffer, and a Tris buffer.

In the method, the N₂O or NO may be derived from a waste gas or a wastewater. Therefore, according to the method, it is possible to reduce the concentration of N₂O or NO in waste gas or wastewater.

A method of reducing a concentration of N₂O or NO in a medium, according to an aspect, may efficiently reduce the concentration of N₂O or NO in the medium.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a graph of relative N₂O retention rate (percent, %) versus test sample, which shows the relative retention rate of N₂O in a gas layer in a glass serum bottle after culturing Paracoccus versutus in a N₂O-containing medium in the presence or absence of magnesium ions;

FIG. 2 is a graph of relative retention rate (%) versus test sample, which shows the relative retention rates of NO, N₂O, and N₂ in a gas layer in a glass serum bottle after culturing Paracoccus versutus in a Fe(II)EDTA-NO-containing medium in the presence or absence of magnesium ions;

FIG. 3 is a graph of relative N₂O retention rate (%) versus test sample, which shows relative retention rates of N₂O in a gas layer in a glass serum bottle after culturing Paracoccus versutus in a 2.5 mM N₂O-containing medium in the presence or absence of Mg²⁺, Ca²⁺, Mn²⁺, Mo²⁺, Cu²⁺, or Co²⁺ ions; and

FIG. 4 is a graph of relative retention rate (%) versus test sample, which shows the relative retention rates of NO, N₂O, and N₂ in a gas layer in a glass serum bottle after culturing Paracoccus versutus or Pseudomonas stutzeri in a Fe(II)EDTA-NO-containing medium in the presence or absence of magnesium ions.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

As used herein, the term “and/or” includes any and all combinations of one or more of the associated listed items. Expressions such as “at least one of,” when preceding a list of elements, modify the entire list of elements and do not modify the individual elements of the list.

The terminology used herein is for the purpose of describing particular embodiments only and is not intended to be limiting. “At least one” is not to be construed as limiting “a” or “an.” As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Some microorganisms of the genus Pseudomonas or the genus Paracoccus are known to biologically reduce nitrogen oxides. A denitrification process using these microorganisms makes it possible to remove almost all nitrates and nitrites. However, in such a biological denitrification process, it is difficult to convert N₂O and NO compounds into molecular nitrogen, and therefore, it is difficult for a treatment system to completely remove them.

Hereinafter, the present disclosure will be described in more detail with reference to exemplary embodiments. However, these exemplary embodiments are only for illustrating the present disclosure, and the scope of the present disclosure is not limited to these exemplary embodiments.

### Example 1: Effect of Magnesium Ions on N₂O Removal by Paracoccus versutus

In this exemplary embodiment, the effect of magnesium ions on N₂O removal by the bacterium Paracoccus versutus was examined. In detail, Paracoccus versutus was primarily cultured in a 250 milliliter (ml) Erlenmeyer flask containing an LB medium (10 grams per liter (g/L) Tryptone, 10 g/L NaCl, 5 g/L Yeast Extract), and then cells of the cultured microorganism were isolated by centrifugation. Next, the isolated cells were washed with an M9 medium (6.78 g/L of Na₂HPO₄, 3 g/L of KH₂PO₄, 0.5 g/L of NaCl, 1 g/L of NH₄Cl, 0.1 mg/L of MnSO₄, 0.1 mg/L of Na₂MoO₄, 0.1 mg/L of CuSO₄ 5H₂O, and 2 mg/L of CaCl₂). The washed cells were mixed with 30 ml of M9 medium in a 60 ml glass serum bottle, i.e., a serum bottle sealed with a silicone stopper and an aluminum cap, so that the cell concentration in the medium was resulted in an optical density (O.D.) of 1 (OD=1). Then, 2.5 millimolar (mM) N₂O was injected using a syringe into the glass serum bottle containing the cells and the M9 medium, and the serum bottle was sealed with a silicone stopper and an aluminum cap, followed by incubation of the serum bottle at 30° C. for 7 hours with shaking. The upper gas layer in the glass serum bottle was collected, and the concentration of N₂O or N₂ was measured by gas chromatography mass-spectrometry (GC-MS). In this regard, an M9 medium containing no cells was used as a negative control group, an M9 medium containing cells without magnesium ions was used as a positive control group, and an M9 medium containing 1 mM magnesium ions and cells was used as an experimental group.

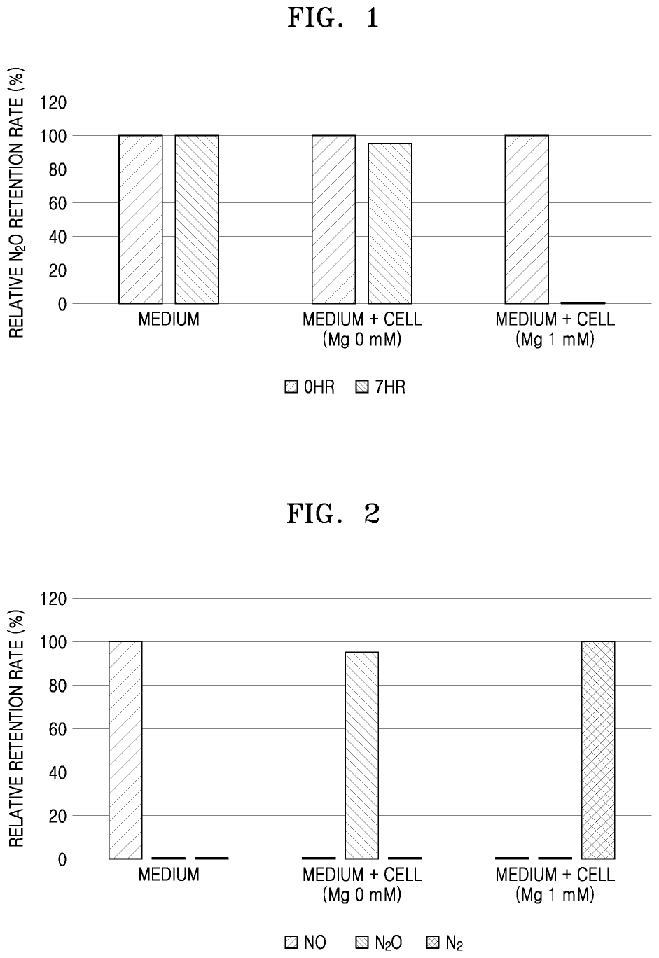

FIG. 1 shows a relative retention rate of N₂O in the gas layer present in the glass serum bottle after culturing the Paracoccus versutus in the N₂O-containing medium in the presence or absence of magnesium ions. The relative retention rate was calculated according to the following equation.

Relative retention rate (%)=(Remaining amount/Initial injection amount)×100%

Table 1 is a table showing the conversion rate of N₂O to N₂, through the reduction of N₂O and the production of N₂ in the gas layer in the serum bottle after culturing Paracoccus versutus in the N₂O-containing medium in the presence, or absence, of magnesium ions.

Conversion rate (%)=((initial injection amount−remaining amount)/initial injection amount)×100%

TABLE 1

| | M9 | 0 mM Mg²⁺- | 1 mM Mg²⁺- |
|---|---|---|---|
| | medium | containing M9 | containing M9 |
| Sample | (no cells) | medium + cells | medium + cells |
| N₂ | 0 | 0 | 100 |
| conversion | | | |
| rate (%) | | | |

As shown in Table 1, when Paracoccus versutus was cultured in the M9 medium without Mg²⁺ (0 mM Mg²⁺-containing M9 medium), the conversion rate of N₂O to N₂ was 0%. In contrast, when Paracoccus versutus was cultured in the M9 medium with Mg²⁺ (1 mM Mg²⁺-containing M9 medium), the conversion rate of N₂O to N₂ was 100%. This indicates that when Paracoccus versutus was cultured in the M9 medium with Mg²+, 100% of the N₂O was converted to N₂.

### Example 2: Effect of Magnesium Ions on NO Removal by Paracoccus versutus

In this exemplary embodiment, the effect of magnesium ions on NO removal by the bacterium Paracoccus versutus was examined. In detail, Paracoccus versutus was primarily cultured in a 250 ml Erlenmeyer flask containing an LB medium (10 g/L Tryptone, 10 g/L NaCl, 5 g/L Yeast Extract), and then cells of the microorganism were isolated by centrifugation. Next, the isolated cells were washed with an M9 medium (6.78 g/L of Na₂HPO₄, 3 g/L of KH₂PO₄, 0.5 g/L of NaCl, 1 g/L of NH₄Cl, 0.1 mg/L of MnSO₄, 0.1 mg/L of Na₂MoO₄, 0.1 mg/L of CuSO₄ 5H₂O, 2 mg/L of CaCl₂)), and the washed cells were mixed with 30 ml of M9 medium containing 5 mM ferrous ethylenediaminetetraacetate-nitric oxide (Fe(II)EDTA-NO) in a 60 ml glass serum bottle, i.e., a serum bottle sealed with a silicone stopper and an aluminum cap, so that the cell concentration in the medium resulted in an OD of 1. Then, the glass serum bottle, which contained the cells, the Fe(II)EDTA-NO, and the M9 medium, was sealed with the silicone stopper and the aluminum cap, and incubated at 30° C. for 7 hours with shaking. The upper gas layer in the glass serum bottle was collected, and the concentration of NO, N₂O and N₂ was measured by gas chromatography mass-spectrometry (GC-MS).

In this regard, a Fe(II)EDTA-NO-containing M9 medium without cells was used as a negative control group, a Fe(II)EDTA-NO-containing M9 medium with cells and without magnesium ions was used as a positive control group, and a Fe(II)EDTA-NO-containing M9 medium with 1 mM magnesium ions and cells was used as an experimental group.

FIG. 2 shows relative retention rates of NO, N₂O, and N₂ in the gas layer in the glass serum bottle after culturing Paracoccus versutus in the Fe(II)EDTA-NO-containing medium in the presence or absence of magnesium ions. The relative retention rate was calculated according to the following equation.

Relative retention rate (%)=(Remaining amount/Initial injection amount)×100%

Table 2 is a table showing the values of the relative retention rates of NO, N₂O, and N₂ in the gas layer in the serum bottle after culturing Paracoccus versutus in the Fe(II)EDTA-NO-containing medium in the presence or absence of magnesium ions.

TABLE 2

| | Medium | | | 0 mM Mg²⁺-containing | | | 1 mM Mg²⁺-containing | | |
|---|---|---|---|---|---|---|---|---|---|
| | (no cells) | | | medium + cells | | | medium + cells | | |
| Sample | NO | N₂O | N₂ | NO | N₂O | N₂ | NO | N₂O | N₂ |
| Relative retention | 100 | 0 | 0 | 0 | 100 | 0 | 0 | 0 | 100 |
| rate (%, 24 hr later) | | | | | | | | | |

As shown in Table 2, when culturing was performed without Paracoccus versutus cells in the M9 medium without Mg²⁺, only NO was present, and the conversion rate of NO to either N₂O or N₂ was 0%. When Paracoccus versutus was cultured in the M9 medium with 0 mM Mg²⁺, conversion of NO into N₂O was 100%, and no conversion of N₂O into N₂ occurred. In contrast, when Paracoccus versutus was cultured in the M9 medium with 1 mM Mg²+, 100% of NO was converted to N₂.

### Example 3: Effect of Divalent Cations on N₂O Removal by Paracoccus versutus

In this exemplary embodiment, the effects of magnesium ions and five other divalent cations on NO denitrification by the bacterium Paracoccus versutus were examined. In detail, Paracoccus versutus was primarily cultured in a 250 ml Erlenmeyer flask containing an LB medium (10 g/L Tryptone, 10 g/L NaCl, 5 g/L Yeast Extract), and then cells of the microorganism were isolated by centrifugation. Next, the isolated cells were washed with an M9 medium (6.78 g/L of Na₂HPO₄, 3 g/L of KH₂PO₄, 0.5 g/L of NaCl, 1 g/L of NH₄Cl, 0.1 mg/L of MnSO₄, 0.1 mg/L of Na₂MoO₄, 0.1 mg/L of CuSO₄ 5H₂O, 2 mg/L of CaCl₂), and the washed cells were mixed with 30 ml of M9 medium in a 60 ml glass serum bottle, i.e., a serum bottle sealed with a silicone stopper and an aluminum cap, so that the cell concentration in the medium resulted in OD of 1. Then, 2.5 mM N₂O was injected using a syringe into the glass serum bottle which contained the cells and the medium, and the glass serum bottle was sealed with a silicone stopper and an aluminum cap, followed by culturing at 30° C. for 7 hours with shaking. The upper gas layer in the glass serum bottle was collected, and the concentration of N₂O or N₂ was measured by gas chromatography mass-spectrometry (GC-MS).

In this regard, an M9 medium containing no cells was used as a negative control group, and an M9 medium containing 1 mM of Mg²⁺, Ca²⁺, Mn²⁺, Mo²⁺, Cu²⁺ or Co²⁺ ions and cells was used as an experimental group.

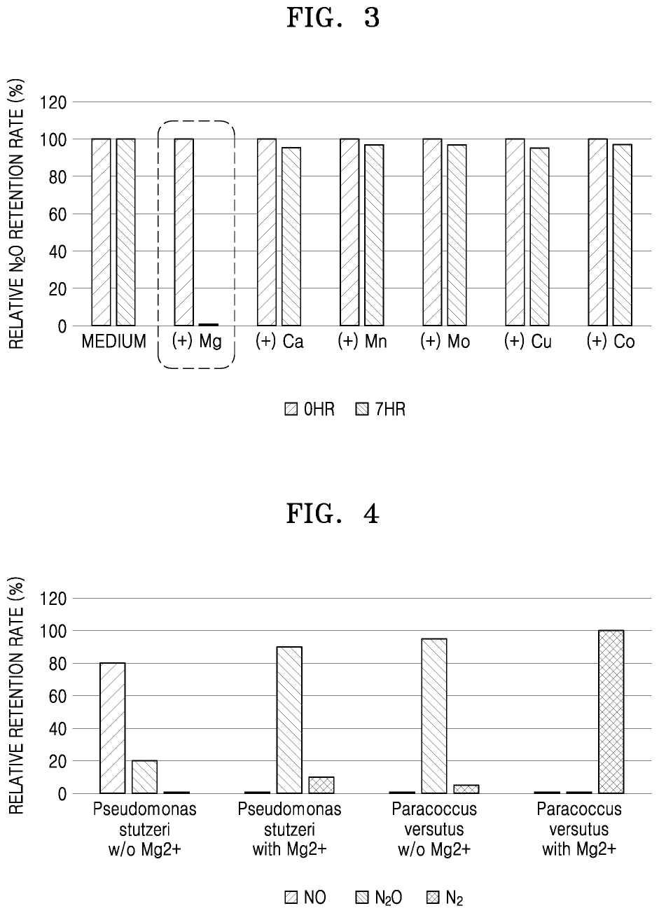

FIG. 3 shows the relative N₂O retention rates of N₂O and N₂ in the gas layer in the glass serum bottle after culturing Paracoccus versutus in the presence of 2.5 mM N₂O-containing medium in the presence or absence of Mg²⁺, Ca²⁺, Mn²⁺, Mo²⁺, Cu²⁺, or Co²⁺ ions. The relative retention rate was calculated according to the following equation.

Relative retention rate (%)=(Remaining amount/Initial injection amount)×100%

Table 3 is a table showing the relative retention rates of N₂O and N₂ in the gas layer in the serum bottle after culturing Paracoccus versutus in the N₂O-containing medium in the presence or absence of Mg²⁺, Ca²⁺, Mn²⁺, Mo²⁺, Cu²⁺ or Co²⁺ ions.

TABLE 3

| | Medium | | Mg²⁺ | | Ca²⁺ | | Mn²⁺ | | Mo²⁺ | | Cu²⁺ | | Co²⁺ | |
|---|---|---|---|---|---|---|---|---|---|---|---|---|---|---|
| Medium | N₂O | N₂ | N₂O | N₂ | N₂O | N₂ | N₂O | N₂ | N₂O | N₂ | N₂O | N₂ | N₂O | N₂ |
| Retention rate | 100 | 0 | 0 | 100 | 95 | 0 | 97 | 0 | 97 | 0 | 95 | 0 | 96 | 0 |
| (%, 7 hr later) | | | | | | | | | | | | | | |

As shown in Table 3, when Paracoccus versutus cells were cultured in the M9 medium with Ca²⁺, Mn²⁺, Mo²⁺, Cu²⁺ or Co²⁺ ions, only N₂O was present, and the conversion rate of N₂O to N₂ was 0%. When Paracoccus versutus was cultured in the M9 medium with 1 mM Mg²+, 100% of N₂O was converted to N₂.

### Example 4: Effect of NO Removal by Paracoccus versutus or Pseudomonas stutzeri

In this exemplary embodiment, the effect of magnesium ions on NO removal by Paracoccus versutus or Pseudomonas stutzeri (purchased from Biological Resource Center, KCTC) was examined. In detail, Paracoccus versutus or Pseudomonas stutzeri was primarily cultured in a 250 ml Erlenmeyer flask containing an LB medium (10 g/L Tryptone, 10 g/L NaCl, 5 g/L Yeast Extract), and then the cells of the microorganism were isolated by centrifugation. Next, the isolated cells were washed with an M9 medium (6.78 g/L of Na₂HPO₄, 3 g/L of KH₂PO₄, 0.5 g/L of NaCl, 1 g/L of NH₄Cl, 0.1 mg/L of MnSO₄, 0.1 mg/L of Na₂MoO₄, 0.1 mg/L of CuSO₄ 5H₂O, 2 mg/L of CaCl₂), and the washed cells were mixed with 30 ml of 5 mM Fe(II)EDTA-NO-containing M9 medium in a 60 ml glass serum bottle, i.e., a serum bottle sealed with a silicone stopper and an aluminum cap, so that the cell concentration in the medium resulted in an OD of 1. Then, the glass serum bottle containing the cells, Fe(II)EDTA-NO, and the medium, was sealed with a silicone stopper and an aluminum cap, and incubated at 30° C. for 7 hours with shaking. The upper gas layer in the glass serum bottle was collected, and the concentration of NO, N₂O, and N₂ was measured by gas chromatography mass-spectrometry (GC-MS).

FIG. 4 shows the relative retention rates of NO, N₂O, and N₂ in the gas layer in the serum bottle after culturing Paracoccus versutus or Pseudomonas stutzeri in the Fe(II)EDTA-NO-containing medium in the presence or absence of magnesium ions. The relative retention rate was calculated according to the following equation.

Relative retention rate (%)=(Remaining amount/Initial injection amount)×100%

As shown in FIG. 4, when culturing was performed without Paracoccus versutus cells in the M9 medium without Mg²⁺, most of the NO was converted to N₂O, and conversion to N₂ was low. When Paracoccus versutus was cultured in the M9 medium with 1 mM Mg²⁺, conversion of NO into N₂ was 100%. In contrast, when Pseudomonas stutzeri cells were cultured in the M9 medium without Mg²⁺, about 20% of NO was converted to N₂O, and NO was hardly converted to N₂. When Pseudomonas stutzeri cells were cultured in the M9 medium with 1 mM Mg²⁺, conversion of NO into N₂O or N₂ was 100%. This indicates that Mg²⁺ ions contribute to nitric oxide decomposition also in the Pseudomonas stutzeri strain.

It should be understood that embodiments described herein should be considered in a descriptive sense only and not for purposes of limitation. Descriptions of features or aspects within each embodiment should be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of reducing a concentration of N₂O, NO, or a combination thereof in a medium, the method comprising:
culturing a microorganism of the genus Paracoccus, a microorganism of the genus Pseudomonas, or a combination thereof in a liquid medium comprising Mg²⁺ ions and Fe(II)(L)-NO, N₂O, or a combination thereof, wherein L is a chelating agent; and
reducing NO to N₂O or N₂, or reducing N₂O to N₂.

2. The method of claim 1, wherein the microorganism of the genus Paracoccus comprises Paracoccus versutus, Paracoccus denitrificans, Paracoccus pantothrophas, Paracoccus ferrooxidans, Paracoccus denitrificans, or a combination thereof.

3. The method of claim 1, wherein the microorganism of the genus Pseudomonas comprises Pseudomonas stutzeri, Pseudomonas putida, Pseudomonas cepacia, Pseudomonas fluorescens, Pseudomonas mendocina, or a combination thereof.

4. The method of claim 1, wherein the liquid medium comprises about 0.1 millimolar to about 7.5 millimolar of Mg²⁺ ions.

5. The method of claim 1, wherein the L is ethylenediamine, diethylenetriamine, triethylenetetraamine, hexamethylenetetraamine, N-(2-hydroxyethyl)ethylenediamine-triacetic acid, ethylenediamine-tetraacetic acid, iminodiacetic acid, nitrilotriacetic acid, or diethylenetriaminepentaacetic acid.

6. The method of claim 1, wherein the liquid medium enables growth of the microorganism of the genus Paracoccus or growth of the microorganism of the genus Pseudomonas.

7. The method of claim 1, wherein the culturing is performed under anaerobic conditions.

8. The method of claim 1, wherein the culturing is performed at a temperature of about 25° C. to about 40° C.

9. The method of claim 1, wherein the culturing comprises culturing of the microorganism of the genus Paracoccus alone, culturing of the microorganism of the genus Pseudomonas alone, or culturing of a mixture of the microorganism of the genus Paracoccus and the microorganism of the genus Pseudomonas.

10. The method of claim 1, wherein the method further comprises forming of Fe(II)EDTA-NO by contacting NO₂ or NO with a liquid medium comprising Fe(II)EDTA, and
the culturing is performed at the same time as the forming of Fe(II)EDTA-NO or after the forming of Fe(II)EDTA-NO.

11. The method of claim 1, wherein the concentration of Fe(II)(L)-NO in the liquid medium is about 0.1 millimolar to about 20 millimolar.

12. The method of claim 1, wherein the liquid medium further comprises an electron donor.

13. The method of claim 12, wherein the electron donor is methanol, ethanol, acetate, glucose, or a combination thereof.

14. The method of claim 1, wherein the liquid medium is a buffer.

15. The method of claim 1, wherein the liquid medium comprises an LB medium, an M9 medium, a phosphate buffer, a Tris buffer, or a combination thereof.

16. The method of claim 1, wherein the N₂O or the NO is derived from a waste gas or a wastewater.
